(19) United States
(12) Patent Application Publication
(10) Pub. No.: US 2010/0030020 A1
(43) Pub. Date: Feb. 4, 2010

(54) Optical surgical device and method of use

(75) Inventors: Gerald J. Sanders, Sonoma, CA (US); Vivek Sikri, Cambridge, MA (US)

Correspondence Address: MINTZ LEVIN COHN FERRIS GLOVSKY & POPEO, ONE FINANCIAL CENTER, BOSTON, MA, 02111, US

(73) Assignee: FemSuite LLC, San Francisco, CA (US)

(21) Appl. No.: 12/456,666

(22) Filed: Jun. 19, 2009

Related U.S. Application Data
(63) Continuation-in-part of application No. 11/975,409, filed on Oct. 19, 2007.
(60) Provisional application No. 61/132,735, filed on Jun. 19, 2008.
(60) Provisional application No. 61/137,350, filed on Jul. 29, 2008.
(60) Provisional application No. 61/207,696, filed on Feb. 12, 2009.
(60) Provisional application No. 60/853,161, filed on Oct. 20, 2006.
(60) Provisional application No. 60/878,892, filed on Jan. 4, 2007.
(60) Provisional application No. 60/903,583, filed on Feb. 26, 2007.
(60) Provisional application No. 60/921,925, filed on Apr. 4, 2007.
(60) Provisional application No. 60/925,486, filed on Apr. 20, 2007.
(60) Provisional application No. 60/933,233, filed on Jun. 4, 2007.

(51) Int. Cl. A61B 1/04

(52) U.S. Cl. 600/109

(57) ABSTRACT

An optical device includes a shaft, a handle and a camera assembly. The handle is coupled to the shaft at a first end, and the camera assembly is coupled to the shaft at a second end. Camera circuitry and software may be provided in the shaft and the handle, so that, in one embodiment, the device may be constructed with reusable portions of the camera circuitry and software.

### CROSS-REFERENCE TO RELATED APPLICATIONS

The present application claims priority to U.S. Provisional Patent Application No. 61/132,735 to Sanders et al., filed Jun. 19, 2008, and entitled “Optical Surgical Device and Methods of Use”, U.S. Provisional Patent Application No. 61/137,350 to Sanders et al., filed Jul. 29, 2008, and entitled “Optical Surgical Device and Methods of Use”, and to U.S. Provisional Patent Application No. 61/207,696 to Sanders et al., filed Feb. 12, 2009, and entitled “Optical Surgical Device and Methods of Use”.

The present application is also a continuation-in-part application of U.S. patent application Ser. No. 11/975,409 to Sanders et al., filed Oct. 19, 2007, and entitled “Optical Surgical Device and Methods of Use”, which claims priority to U.S. Provisional Patent Application Nos. 60/853,161, filed Oct. 20, 2006, 60/878,892, filed Jan. 4, 2007, 60/903,583, filed Feb. 26, 2007, 60/921,925, filed Apr. 4, 2007, 60/925,486, filed Apr. 20, 2007, and 60/933,233, filed Jun. 4, 2007.

The present application incorporates the disclosures of all of the above application by reference herein in their entireties.

### BACKGROUND OF THE INVENTION

1. Field of the Invention

The present invention relates to apparatus and methods for imaging body tissue during medical procedures. More particularly, the present invention relates to apparatus and methods that provide endoscopic viewing of the female genital tract during gynecological procedures.

2. Background of the Invention

A number of gynecological procedures require visual inspection of the genital tract of a female patient, which is often performed with the use of an endoscope, or hysteroscope. Conventional endoscopes are often constructed from a rigid body and oftentimes those rigid bodies include fluid conduits.

For example, colposcopy is a diagnostic procedure, in which a lighted binocular microscope called a colposcope is utilized to examine an illuminated, magnified view of the vulva, vagina, and cervix. Most women undergo colposcopic examination to investigate abnormalities in their pap smears, or to assess diethylstilbestrol (DES) exposure in utero, HIV infection or immunosuppression. The enlarged view provided by a colposcope enables a clinician to visually distinguish normal from abnormal appearing tissue and take directed biopsies for pathological examination.

Colposcopy is performed with the patient in the dorsal lithotomy position, in which the patient lies with her legs in stirrups and her buttocks close to the lower edge of the examining table. A speculum is placed in the vagina after the vulva is examined for any suspicious lesions, and an acetic acid solution (e.g., Lugol's or Schiller's) is applied to the cervix to improve visualization and to help the clinician assess whether a change in color or in the vascular pattern of the patient are indicative of abnormalities. After a complete examination, the clinician determines the areas with the highest degree of visual abnormality and obtains biopsies from these areas using a long biopsy instrument.

Colposcopy is an expensive procedure that requires a dedicated instrument, the colposcope, and a specially trained clinician. While colposcopy is considered a preferred procedure for diagnosing cervical abnormalities, it also has some drawbacks. The cost of the colposcope and of the clinical training required to perform it limit application. Additionally, the colposcope is a bulky instrument, usable only in dedicated clinical settings, and provides no view of the uterus. Due to the nature of the colposcope, separate instruments must be employed for taking biopsies and, when required, for endocervical curettage (ECC).

The uterine cavity may be examined by hysteroscopy, which is a diagnostic procedure that enables a clinician to diagnose intrauterine pathology and which may provide a method for surgical intervention (operative hysteroscopy).

Hysteroscopy is performed with an endoscopic device, called a hysteroscope. Some hysteroscopes include a stiff shaft coupled to a handle, a vision member at the tip of the shaft connected to fiber optics and to a video system, and a channel for delivering a distention medium. Because the uterus is a potential cavity, it is first distended either with a fluid (saline, sorbitol, or a dextrane solution) or a gas (CO2), and the stiff shaft carrying the vision member is introduced in the uterus through the cervical canal.

Different types of hysteroscopes may be used for different gynecological interventions. While the hysteroscope is typically a viewing device only, an operative hysteroscope includes a working channel that allows specialized instruments to enter the uterine cavity and perform surgery, and a resectoscope is a variation of the hysteroscope that contains an electric loop for resecting a submucous leiomyoma.

Hysteroscopy has been found useful to treat a variety of uterine conditions, such as polyps, leiomyomata, Asherman syndrome, gynecologic bleeding, and uterine malformations, but occasionally a uterine perforation occurs when the stiff shaft breaches the wall of the uterus leading to bleeding and to damage to other organs. Another drawback of known hysteroscopes is limited maneuverability, due to the rigidity of the shaft that makes it difficult to maneuver the instrument within the patient's genital system. Still other drawbacks relate to the use of fiber optics, which are manufactured from glass that breaks under bending stress, requiring frequent maintenance of the hysteroscope with consequent downtime and costs. Additionally, in known hysterocopes the camera, saline channel, and working channel all have distal openings at the distal tip of the shaft, causing an increase in the diameter of the tip and making the instrument more invasive to the patient. A corresponding decrease in channel diameters decreases the efficiency of the instrument and makes it more difficult to clean and sterilize.

Attempts have been made to remedy these drawbacks of conventional hysteroscopes. For example, U.S. Pat. No. 4,836,189 to Allred, III et al. describes a video hysteroscope having an elongated flexible insertion tube containing a video member at its distal end, as well as a channel for a surgical laser fiber and a saline channel that emits a continuous stream of saline solution. An articulation section joins the viewing head to a flexible tubular member.

U.S. Pat. No. 5,823,940 to Newman discloses a sheath that receives an endoscope. In that device, the endoscope includes a bundle of fiber optics that is slid into a lumen in the sheath. The sheath is flexible and includes additional fluid conduits. After a procedure is performed with the sheath, the endoscope is removed from the sheath, which is then discarded.

U.S. Publication No. 2005/0288551 to Callister et al. discloses an endoscopic assembly having a flexible hysteroscope and an outer sheath disposed about a length of the shaft of the hysteroscope. An inflatable balloon seals the assembly within a body lumen or cavity.

A drawback of these devices is that although some of them contain disposable components, the hysteroscope and an associated eyepiece still require cleaning and sterilization, contain fragile fiber optics, and have tips with sizes that make the instruments uncomfortable or even painful when traveling through the cervical canal and into the uterus, and correspondingly limit the diameters of the lumens in the instrument. Another drawback is that the light colors provided by some of these instruments are within a limited palette, while different types of anomalies are better viewable with different light combinations.

It would be therefore be desirable to provide improved apparatus and methods for inspecting body tissues that, in various embodiments, remedy some or all of the aforementioned drawbacks of previous optical devices.

### SUMMARY OF THE INVENTION

In some embodiments, the present invention relates to an optical apparatus for examining a female genital tract. The device includes a housing including a shaft portion, and a handle portion extending from a proximal end of the shaft portion and defining a cavity that contains an output connector, a camera assembly coupled to a distal end of the shaft, the camera assembly comprising a camera, and a removable cartridge having a limited number of uses and is receivable within the cavity and including an input connector matable with the output connector, an image processing engine, a power source, a light source, and a heat-absorption device for absorbing heat generated by the light source, wherein the light source is configured to direct light toward the input connector and illuminate a field of view for the camera.

In some embodiments, the present invention relates to an optical apparatus. The apparatus includes a housing including a shaft portion, and a handle portion extending from a proximal end of the shaft portion and defining a cavity that contains an output connector; a camera assembly coupled to a distal end of the shaft, the camera assembly comprising a camera; and a removable cartridge having a limited number of uses and is receivable within the cavity. The cartridge includes an input connector matable with the output connector; an image processing engine; a power source; a light source; a heat-absorption device for absorbing heat generated by the light source; a heat-monitoring device for monitoring temperature of the heat-absorption device during operation of the removable cartridge. The light source is configured to direct light toward the input connector and illuminate a field of view for the camera.

In some embodiments, the present invention relates to a method for examining a bodily cavity using an optical apparatus described above. The method includes the steps of using the light source, examining the bodily cavity; using the heat absorption device, absorbing heat generated by the light source during examination of the bodily cavity; determining a temperature of the heat absorption device; comparing the determined temperature to a predetermined threshold temperature; and turning off the light source when the determined temperature exceeds the predetermined threshold temperature.

In some embodiments, the present invention relates to an optical apparatus. The apparatus includes a housing including a shaft portion, and a handle portion extending from a proximal end of the shaft portion and defining a cavity that contains an output connector; a distal tip portion configured to be pivotally coupled to a distal end of the shaft portion; a first camera assembly coupled to a distal tip of the shaft, the first camera assembly comprising a first camera and a first light source, wherein the first camera assembly is disposed at a distal end of the distal tip; a second camera assembly coupled to a distal tip of the shaft, the second camera assembly comprising a second camera and a second light source, wherein the second camera assembly is disposed in a sidewall of the distal tip. The first and second light source are configured to illuminate respective fields of view for the first and second camera.

In some embodiments, the present invention relates to a method for examining a bodily cavity using an optical apparatus described above. The method includes the steps of inserting the device into a bodily cavity; pivoting the distal tip away from the shaft portion; and, using at least one or both of the first and second light sources and at least one or both of first and second cameras, examining the bodily cavity.

In some embodiments, the present invention relates to a optical apparatus. The apparatus includes a housing including a shaft portion, and a handle portion extending from a proximal end of the shaft portion and defining a cavity that contains an output connector; a distal tip portion configured to be removably coupled to a distal end of the shaft portion; a camera assembly coupled to the distal tip of the shaft, the camera assembly comprising a camera and a light source, wherein the camera assembly is disposed at a distal end of the distal tip; wherein the light source is configured to illuminate a field of view for the camera.

In some embodiments, the present invention relates to a method for examining a bodily cavity using an optical apparatus described above. The method includes the steps of: inserting the device into a bodily cavity; extending the distal tip away from the shaft portion; and, using the light source and the camera, examining the bodily cavity.

### BRIEF DESCRIPTION OF THE DRAWINGS

The foregoing summary is only illustrative of the inventions disclosed herein. Further features of the invention, its nature and various advantages will be more apparent from the accompanying drawings and the following detailed description of the preferred embodiments.

FIG. 1 illustrates an exemplary apparatus for endoscopic viewing of a female genital tract during gynecological procedure, according to some embodiments of the present invention.

FIGS. 2a-b illustrate an exploded view of the apparatus shown in FIG. 1.

FIGS. 3a-b illustrate exploded views of an exemplary cartridge disposed in the apparatus shown in FIG. 1, according to some embodiments of the present invention.

FIG. 4a is an exploded view of the exemplary bulkhead of the cartridge shown in FIGS. 3a-b, according to some embodiments of the present invention.

FIG. 4b is an exploded view of an exemplary lid for the cartridge shown in FIGS. 3a-b, according to some embodiments of the present invention.

FIG. 5 illustrates an exemplary configuration of internal components of the tip of the apparatus shown in FIG. 1, according to some embodiments of the present invention.

FIGS. 6 and 7 are flow charts illustrating exemplary visualization/sterilization procedures, according to some embodiments of the present invention.

FIG. 8 is a block diagram of an exemplary diagnostic system that can be configured to perform a thorough diagnostic check on the circuitry within the cartridge of the apparatus shown in FIG. 1, according to some embodiments of the present invention.

FIG. 9 is a flow chart illustrating an exemplary high level diagnostic procedure for the cartridge of the apparatus shown in FIG. 1, according to some embodiments of the present invention.

FIG. 10 is a flowchart illustrating an I2C control testing procedure, according to some embodiments of the present invention.

FIG. 11 is a flow chart of an exemplary NovoSeal control circuit testing procedure, according to some embodiments of the present invention.

FIGS. 12a-b illustrate an alternative embodiment of a light source using an integrating sphere disposed within the cartridge shown in FIGS. 3a-b, according to some embodiments of the present invention.

FIG. 13 is a block diagram of an exemplary heat absorption system, according to some embodiments of the present invention.

FIG. 14 is a flowchart of a method for absorbing heat using the system shown in FIG. 13, according to some embodiments of the present invention.

FIGS. 15a-d illustrate an exemplary tip of the apparatus shown in FIG. 1, according to some embodiments of the present invention.

FIGS. 16a-d illustrate another exemplary tip of the apparatus shown in FIG. 1, according to some embodiments of the present invention.

### DETAILED DESCRIPTION OF THE INVENTION

The present invention relates to apparatus and methods for imaging body tissue during medical procedures. In particular, the present invention relates to apparatus and methods that provide endoscopic viewing of the female genital tract during gynecological procedures.

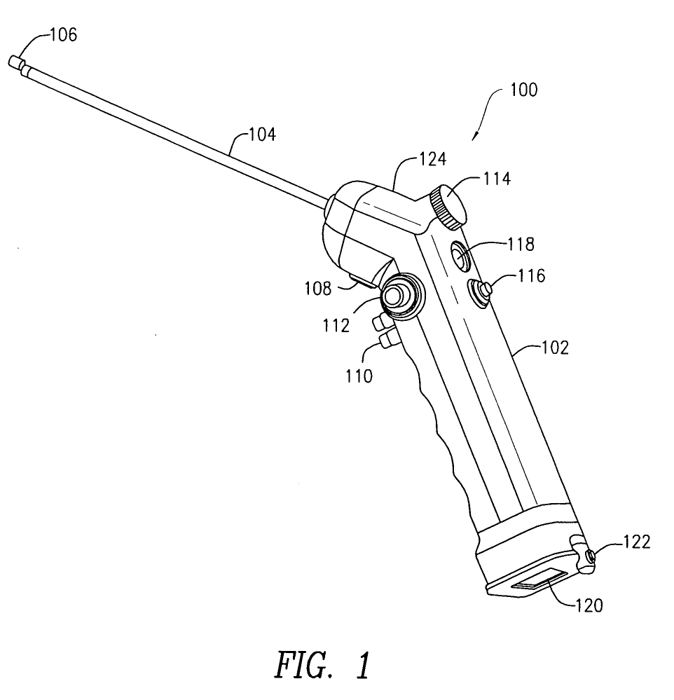

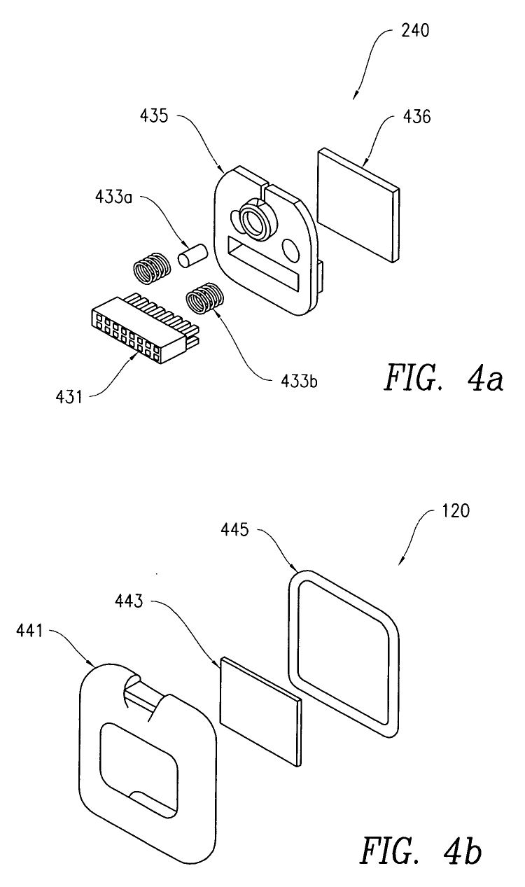

FIG. 1 illustrates an exemplary apparatus 100 for endoscopic viewing of a female genital tract during gynecological procedure, according to some embodiments of the present invention. The apparatus 100 includes a handle housing 102, a shaft 104, and a tip 106. The housing 102 is coupled to the shaft 104. The tip 106 is coupled to the shaft 104. The housing 102 is a hollow housing that is configured to enclose a plurality of components having input and/or output components disposed on an outer surface of the housing 102. The housing 102 includes a closing lid 120 that is pivotally secured via a pivot 122. The lid 120 is configured to secure interior components of the apparatus 100. An exemplary configuration of the lid 120 is shown in FIG. 4b. Referring to FIG. 4b, the lid 120 includes a lid portion 441, a lid switch cover 443, and a lid gasket 445. The gasket 445 is configured to seal the interior portion of the housing 102 once the lid is closed. The lid 120 also includes a hinge portion that secures the lid to the housing 102 and allows the lid 120 to pivot about the hinge portion.

Referring back to FIG. 1, the housing 102 further includes a tip control knob 114, a sterilization catheter connector 112, power/video cable connectors 110, a saline connector 108, an actuator button 116, a working channel entrance 118. As illustrated in FIG. 1, the housing 102 includes two power/video cable connectors 110; in some embodiments, the housing 102 can include a single power/video cable connector 110 for providing power input and video output in a single cable/connector. In some embodiments, the connectors can include strain relievers to prevent excessive bending and/or breaking of the wires for supplying power/video. The housing 102 can also include an LED indicator 124 for indicating status of procedure being performed (e.g., sterilization procedure). The working channel entrance 118 allows insertion of catheters (e.g., sterilization, or any other catheters) for performing procedures on the patient once the device is inserted into the uterus. To maintain seal on the entrance 118, the entrance 118 can be provided with a duckbill valve to properly seal the entrance upon insertion and removal of catheters.

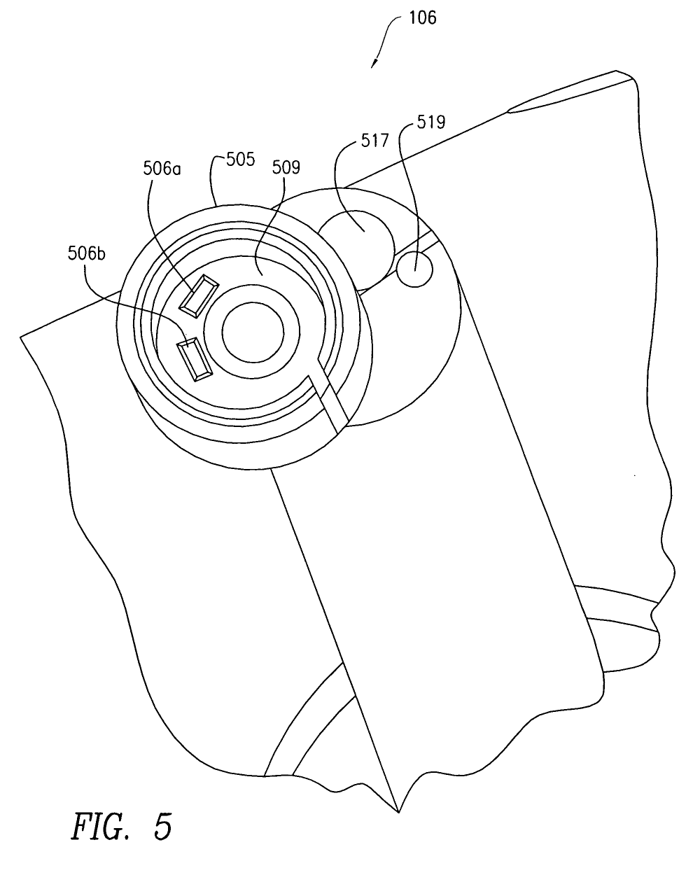

The tip control knob 114 is configured to control opening and closing the tip 106. The tip control knob 114 can be configured to include a pull wire (not shown in FIG. 1) running through the shaft 104 that is connected to the tip 106. Upon rotation of the tip control knob 114, the pull wire is tensioned and the tip 106 is rotated to open up its internal components, such as a camera and a light source (e.g., an LED). (not shown in FIG. 1). An exemplary configuration of the internal components of the tip 106 is illustrated in FIG. 5.

Referring to FIG. 5, the tip 106 is coupled to the shaft 106 and is configured to pivot about a hinged portion 515 to open interior components of the shaft 106 and during examination of internal cavities of female genital tracts. The tip includes a rotating portion 505, a camera portion 509, LED sources 506a and 506b. The tip is also provided with saline channels 517 and 519 for inflow and outflow of a saline solution (which can be used to clear a field of view for the tip, once it is inserted). The LED source 506 is configured to provide light when the tip is inserted into a fallopian tube of the patient. The camera 509 is configured to provide a visual of the interior of a fallopian tube. An exemplary configuration of the tip is disclosed in the co-owned, co-pending U.S. patent application Ser. No. 11/975,409 to Sanders et al., filed Oct. 19, 2007, and entitled “Optical Surgical Device and Methods of Use”, the disclosure of which is incorporated by reference in its entirety.

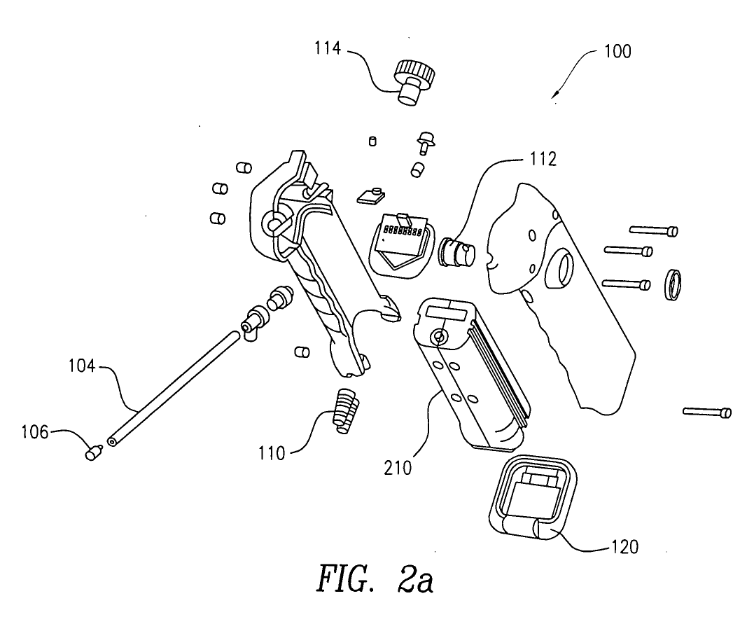

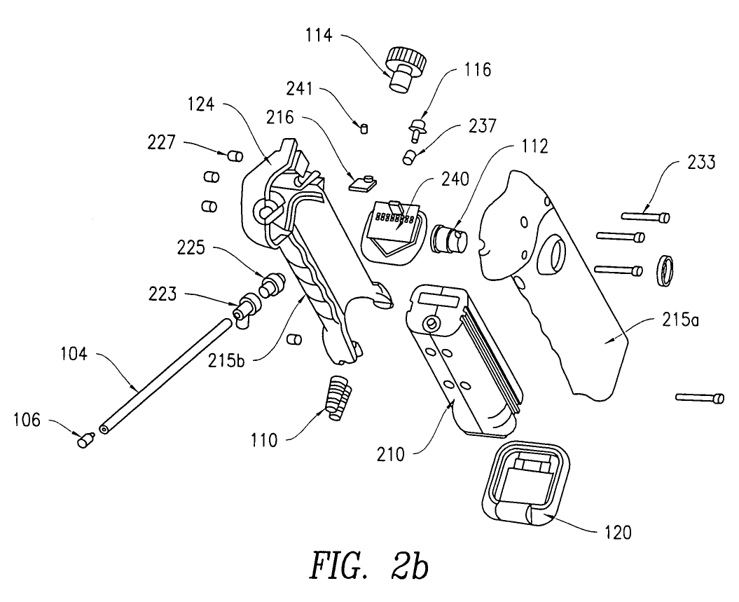

FIGS. 2a-b illustrate an exploded view of the apparatus 100 shown in FIG. 1. The housing 102 includes a first portion 215a and a second portion 215b that can be connected to each other to form an integrated housing 102. The portions 215 can be connected via any locking arrangement, such as a snap-fit arrangement, an interlocking arrangement, or any other method. In some embodiments, connectors 233, 227 can be used to couple portions 215. Connectors 227, 233 can be bolts, screws, snap-fit connectors, or any other suitable connection means. As can be understood by one skilled in the art, the housing 102 can be single-piece housing. The interior space of the housing 102 includes a cartridge 210 for providing power/video to the apparatus 100. The cartridge 210 is secured within the housing 102. In some embodiments, the cartridge 210 can include integrated moldings that are configured to mate with corresponding moldings of the housing 102. The cartridge 210 can be re-loaded (e.g., for the purposes of recharging the power source disposed in the cartridge 210). The lid 120 is configured to prevent accidental removal of the cartridge 210 from the housing 120. The cartridge 210 is coupled to a bulkhead 240.

Referring to FIG. 4a, an exploded view of the exemplary bulkhead 240 is illustrated. The bulkhead 240 serves as a partition wall between connectors and the cartridge 210. It includes a connector portion 431, a partition portion 435, and a printed circuit board (“PCB”) 436. The partition portion 435 includes an opening configured to accommodate insertion of the connector portion 431 for the purposes of connecting it to the PCB 436 via a plurality of connectors. The bulkhead assembly 240 can also include a plurality of springs 433(a, b) configured to interlock the bulkhead 240 to the cartridge 210 (not shown in FIG. 4a). The connectors 431 can be configured to connect the PCB 436 and the cartridge 210 (not shown) to the power/video cables and the tip 106. The PCB 436 is configured to process signals received from the tip 106 and other external components that can be coupled to the apparatus 100 as well as generate signals for controlling operation of the tip 106 and/or other components of the apparatus 100 and/or any other external components connected to apparatus 100. The signals can be received/transmitted via wired connections, wirelessly, or via any other suitable means.

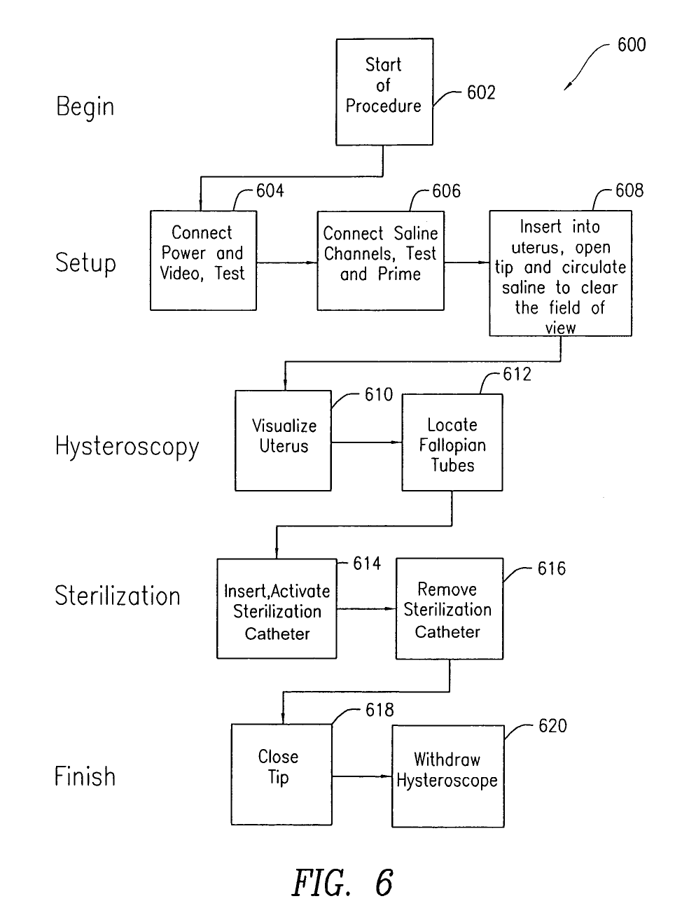

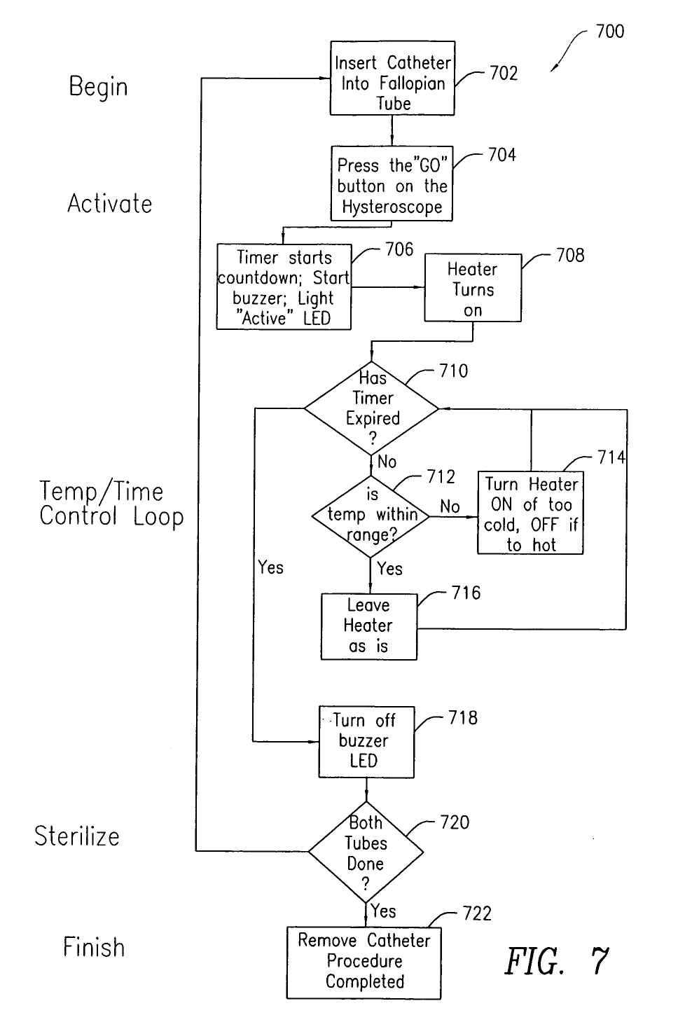

Referring back to FIGS. 2a-b, the shaft 104 is coupled to the housing 102 via various connectors 223 and 225. The connector 225 is coupled to the housing 102 and the connector 225, where the connector 225 is coupled to the shaft 104. The housing portion 215a includes the sterilization catheter connector 112. The portion 215a includes an opening to which the connector 112 is secured. The connector 112 allows connection of a sterilization catheter during a sterilization procedure discussed below. The sterilization procedure can be activated by the user (e.g., a doctor or other medical professional) by pressing the button 116 disposed on the housing 102. The button 116 can be coupled using a spring means 237 to provide an on/off device. The button 116 is also electrically coupled to the electronic components of the apparatus 100, such as the PCB 436 of the bulkhead 240. In some embodiments, the PCB 436 can include a PCB upper component 216. Component 216 is a circuit board having an LED and a detector switch disposed on it. The detector switch is configured to be closed when the rotation knob 114 is turned to open the tip. This allows the microcontroller in the cartridge to determine whether and when the tip is open and when it is closed and thus, whether or not to flip the image upside down. The image is flipped upside down using by sending a command to the image sensor and turning on the auxiliary LED residing in the cartridge. The LED is an indicator light used to provide user feedback during the sterilization procedure. Alternatively, it is configured as an optical warning device indicating that the tip is open. The LED indicator light 124 indicates status of the sterilization procedure, as stated above. Component 241 is a light pipe that allows the LED to shine through the case. In some embodiments, it is configured as a transparent plastic cylinder that is glued in place. FIGS. 6 and 7 discuss visualization/sterilization procedures 600 and 700, respectively.

FIG. 6 illustrates an exemplary visualization/sterilization procedure 600 using the apparatus 100, according to some embodiments of the present invention. The procedure begins by performing certain setup steps (Steps 602, 604, 606, 608). In step 604, the power/video cables are connected and tested. The saline connectors/channels (e.g., for sterilization purposes, etc.) are connected to the apparatus 100 and then are tested and primed. As can be understood by one skilled in the art, testing and priming procedures can be performed using any conventionally known methods. In step 608, the apparatus 100 is inserted into uterus, wherein the tip 106 is inserted first followed by the shaft 104. Then, the tip 106 is opened (using the knob 114 and rotating in one direction, e.g., clockwise or counterclockwise) and the saline solution is circulated to clear the field of view for the tip. The saline solution is received via the saline connectors/channels connected to the apparatus 100. In some embodiments, the saline connectors/channels can include two connectors/channels: one for the inflow of the saline and the other for outflow of the saline. The shaft 104 can also include two channels corresponding to the inflow and outflow connectors/channels. Once the field of view for the tip's cameras is cleared, the method proceeds to hysteroscopy group of steps (steps 610, 612). In step 612, the uterus is visualized using the tip 106. In some embodiments, the tip can use its LED lights to illuminate the uterus and camera to provide video feedback. In step 612, the patient's fallopian tubes are located. Once the tubes are located, the method proceeds to sterilization group of steps (steps 614, 616). In step 614, the sterilization catheter, connected to the apparatus 100 via the sterilization catheter connector 112, is inserted and activated. The catheter 112 can be transported via the shaft 104. As soon as the sterilization procedure is completed, the sterilization catheter is withdrawn, as indicated by step 616. The visualization/sterilization procedure is completed by closing the tip 106 using the knob 114 (e.g., by rotating it in an opposite direction), as indicated by step 618. Then, the apparatus 100 is withdrawn from the uterus.

FIG. 7 illustrates another exemplary visualization/sterilization procedure 700 using the apparatus 100, according to some embodiments of the present invention. The method 700 begins by insertion of the catheter into fallopian tube of the patient, as shown in step 702. Then, the method 700 proceeds to the activation stage that begins with pressing the activation button 116 on the apparatus 100, as shown in step 704. The timer counter in the apparatus 100 begins countdown, indicating the amount of time allotted for a particular procedure (in some embodiments, the apparatus 100 can include a usage counter, which indicates the number of times the apparatus 100 can be used before certain of its components or the entire apparatus need to be replaced, that is configured to increment its value each time the apparatus 100 is used). In some embodiments, the LED indicator 124 can become active (e.g., light up) indicating that visualization/sterilization procedure has begun. Then, the method 700 proceeds to step 708. In step 708, the heater is turned on, which can be used to scar tissue. Then, the method 700 proceeds to the temperature/time control loop (steps 710, 712, 714, 716, 718). In step 710, the apparatus 100 determines whether the timer counter has expired, which would indicate that the time allotted for a particular procedure has expired. If yes, the method proceeds to step 718, where the buzzer and the LED light 124 are turned off. If the time has not expired, the apparatus 100 determines whether the temperature applied by the heater is within a predetermined range/value, as shown in step 712. If not, the heater is turned on if the temperature is below the predetermined range/value or the heater is turned off if the temperature is above the predetermined range/value, as shown in step 714. If the temperature is within the predetermined range/value, then the heater settings are not changed, as indicated in step 716. This loop continues to perform the above checks until the timer counter expires, as indicated by step 710. Once the temperature/time control loop is complete and the buzzer and LED light 124 are turned off indicating completion of the above procedure, the sterilization stage of the method 700 begins (step 720). In step 720, the apparatus 100 determines whether both fallopian tubes of the patient have been examined/treated. If not, the process 700 is repeated beginning with step 702 until both tubes are done. Once both tubes are done, the catheter is removed indicating completion of the visualization/sterilization procedure, as indicated by step 722. The method 700 can be performed using any known heating devices that can be coupled to the apparatus 100.

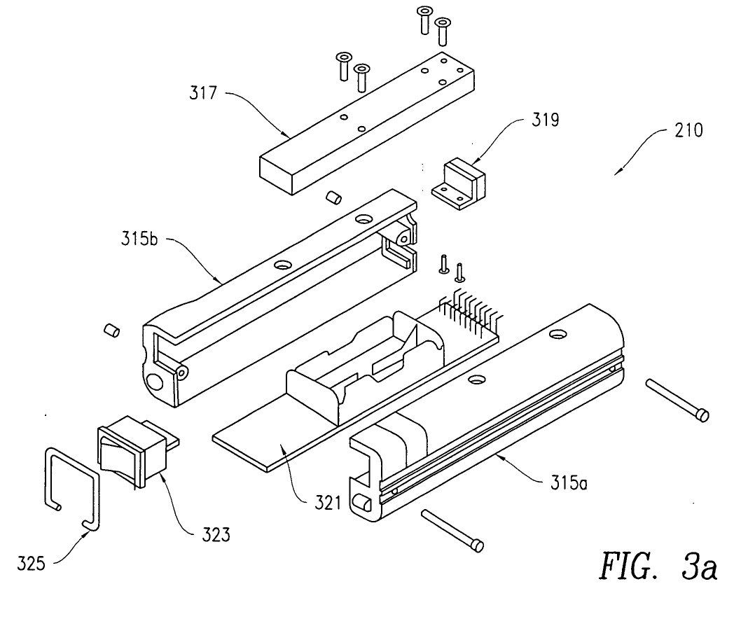

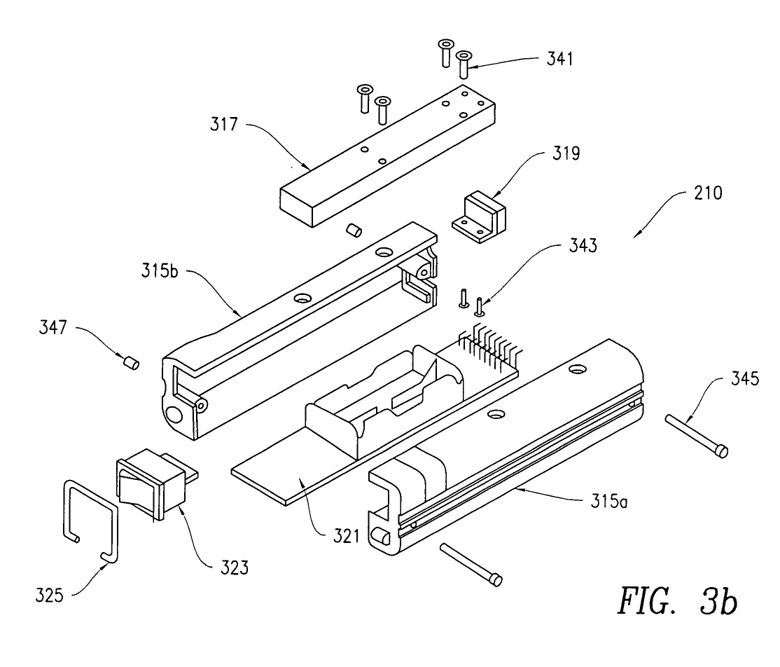

FIGS. 3a-b illustrate exemplary exploded views of the cartridge 210, according to some embodiments of the present invention. The cartridge 210 includes a housing 315 that can include two connectable portions 315a and 315b. The two portions 315a and 315b can be secured together using connectors 345 and 347, which can be bolts, screws, or any other coupling means. The housing 315 is configured to enclose a printed circuit board 321 that includes connectors for connecting to the bulkhead 240 (shown in FIGS. 2a-b) and a heat-sponge plate 317 for absorbing the heat. The plate 317 is secured to the housing 315 using connectors 341, which can be bolts, screws, or any other coupling means. The plate 317 is configured to absorb heat generated by the apparatus 100's LED engine 319. Such heat absorption prevents overheating of the apparatus 100 and meltdown of components. The LED engine 319 is coupled to the PCB 321 and is configured to generate light that is directed toward the fiber coupled to the LED engine 319. The fiber is disposed along the shaft 104 and terminates at the tip 106, thereby illuminating a particular area and providing the camera disposed at the tip 106 (not shown) with an adequate field of view. The intensity of light can be controlled depending on the desired field of view or any other parameters. The housing 315 further includes a switch 323 secured to the housing 315 and electrically coupled to the PCB 321. The switch 323 is configured to control activation of the LED engine 319. The housing 315 also includes a pull ring 325 for pulling the cartridge 210 from the housing 102 of the apparatus 100. The cartridge 210 can be removed after a certain number of uses, which can be preset by the user, manufacturer, or anyone else. In some embodiments, the PCB 321 can include a user counter that determines the number of times that the LED engine 319 has been turned on. In some embodiments, after the LED engine 319 has been used a certain predetermined number of times, a signal can be generated by the counter to indicate that no further uses are available for the LED engine 319 and that it needs to be replaced.

The cartridge 210 can include a power source for providing power to the PCB 321 and the LED engine 319. The power source can be disposed on the PCB 321 or can be separately coupled to the PCB 321. The power source can be rechargeable. In some embodiments, a use counter can be included with the power source that determines the number of times, the power source has been used and/or recharged. Upon reaching a predetermined number of uses and/or recharges, the counter can generate a signal that indicates that no further uses and/or recharges are available and that the power source needs to be replaced with a new power source.

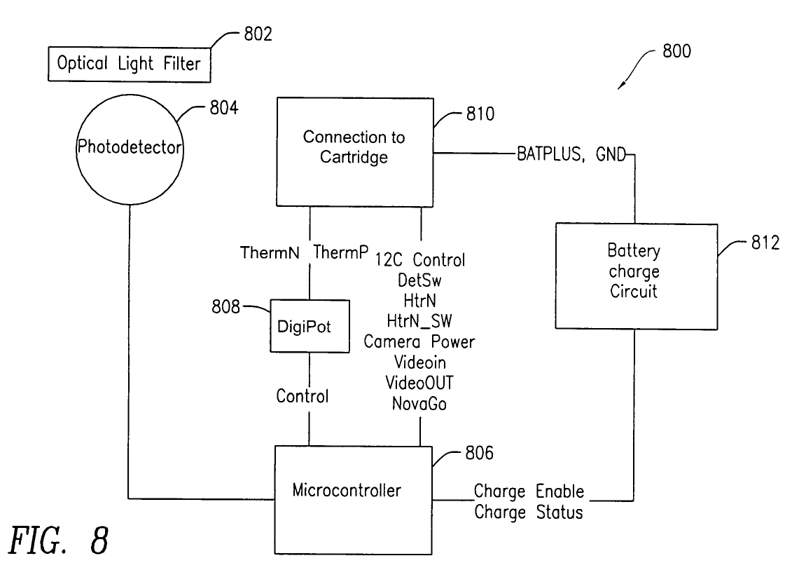

In some embodiments, the cartridge 210 can be recharged using a charger device. An operation of the charger device is discussed below with regard to FIGS. 8-11. In some embodiments, the charger can have three indicator lights:

a. A power light that indicates that the power to the charger is connected;

b. A battery charge status light that has two states:
i. a red state which means that the batter is charging; and
ii. a green state which indicates that the charging is completed;

c. An error indicator light having two states:
i. A solid red state that indicates that the battery will not hold charge, charge count exceeded, or CARTRIDGE ERROR CONDITION;
ii. A blinking red state that indicates that a power switch on the cartridge is in the ON position, or USER ERROR CONDITION;

In some embodiments, the charger can detect the presence of the cartridge 210 and automatically start the charging cycle. The charger can charge the Lithium Ion/Polymer battery in the cartridge 210 or any other battery based on design/requirements for the battery by the user, manufacturer, or any other party. Further, the charger can monitor current and voltage during charging procedure. Additionally, the charger can include an automatic shutoff feature so as to not overcharge the battery. As stated above, the charger can include a user counter to determine the number of times that the batter has been charged. Charger can be configured to adjust (either increment or decrement; for illustrative purposes of the following description, such adjustment will be referred to in terms of incrementing the charger counter) the “charge count” stored in the memory (e.g., EEPROM—a non-volatile) in the cartridge 210. In some embodiments, the memory can be read/written over the serial interface that goes to the camera and as such is accessible via the electrical connector on the cartridge 210. In some embodiments, every time the cartridge is inserted into the charger the “charge count” will be incremented. When the value of “charge count” approaches the number of charge cycles specified, for example, by the battery manufacturer the battery will no longer be charged and the “ERROR” light will indicate a solid red state discussed above.

In some embodiments, the charger can check whether the switch 323 on the cartridge 210 is in the ON position. This can serve as a preventative measure so that the cartridge 210 does not discharge power during its charging cycle. Such procedure can be performed by measuring the voltage on the “Camera Power” pin of the cartridge connector or, alternatively, effectuating a serial interface transaction by simulating an event on the Detector Switch input of the cartridge. Such procedure can be performed using an exemplary diagnostic system 800 illustrated in FIG. 8. The procedure begins by rotating the knob 114 to close the detector switch. The microcontroller in the cartridge 210 is configured to detect closure of the detector switch and, thus, sends a signal to the camera over the serial interface to flip the image upside down. The detector switch closure can be simulated in the charger and based on such simulation, the serial interface of the cartridge can be monitored for any activity. For example, if the cartridge power switch is in the OFF position, the simulation will not generate any effects. However, if the switch is in the ON position, the serial interface will generate some detectable activity.

In some embodiments, the cartridge can include a photodetector to determine LED engine malfunction. For that purpose, an auxiliary light source can be added in the cartridge for the purposes of measuring light output of the LED engine. Additionally, various testing and calibration circuitry can be added to test and calibrate devices coupled to the apparatus 100.

FIG. 8 is a block diagram of an exemplary diagnostic system 800 that can be configured to perform a thorough diagnostic check on the circuitry within cartridge 210 of the apparatus 100, according to some embodiments of the present invention. This allows the user to determine that the cartridge 210 is charged properly as well as functioning properly.

The system 800 includes an optical light filter 802, a photodetector 804, a microcontroller 806, a DigiPot device 808, a connector 810 to the cartridge 210, and a battery charger circuit 812. The optical light filter 802 is a filter that reduces the amount of transmitted light by a predetermined amount. In the embodiments using a very bright auxiliary LED, use of the optical light filter 802 prevents saturation of the photodetector 804 even if it is not within specification. The filter 802 can be configured to optically reduce the amount of light generated by the LED by a factor of 2, 4 or more times. The photodetector 804 is an electronic component capable of measuring the amount of incident light. The cartridge connector 806 is a connector that the cartridge 210 is configured to plug into. The battery charger circuit 812 is configured to charge cartridge power source (e.g., a lithium polymer battery). This circuit can be configured to include an enable signal that comes from the microcontroller 806 and send a charge status signal to the microcontroller 806. The DigiPOT device 808 is an electronically controlled resistance that allows simulation of a thermostat in a catheter that can be inserted through port 118 of the apparatus 100 (not shown in FIG. 8). Changing the resistance across this device emulates different temperatures as far as control circuit(s) in the cartridge 210 is concerned. The microcontroller 806 is configured to control operation of the cartridge 210 during various phases, e.g., diagnostic phase. It can measure the voltage on the camera power line to determine whether the user has the cartridge turned ON or OFF. It can determine whether a heater relay in a heating device coupled to the apparatus 100 in the cartridge is open or closed. It can be an I2C master or slave device as required to test the various functions of the cartridge. It can also check the continuity of the video signal path.

In some embodiments, the above tests can be emulated by performing substitute actions for the actions typically performed by the user during operation of the apparatus 100 and any devices coupled to it. For example, pressing the actuation button 116 can be emulated by setting the corresponding signal high or low as required via software in the microcontroller 806. When the user opens the knob 114, the user closes the contact on a detector switch in the handle 102 of the apparatus 100. This sets a certain signal line to the microcontroller 806 in the cartridge 210 high. The same effect can be achieved by setting that signal line high in the charger's microcontroller which is connected to the signal line in the cartridge which would be set high in normal operation.

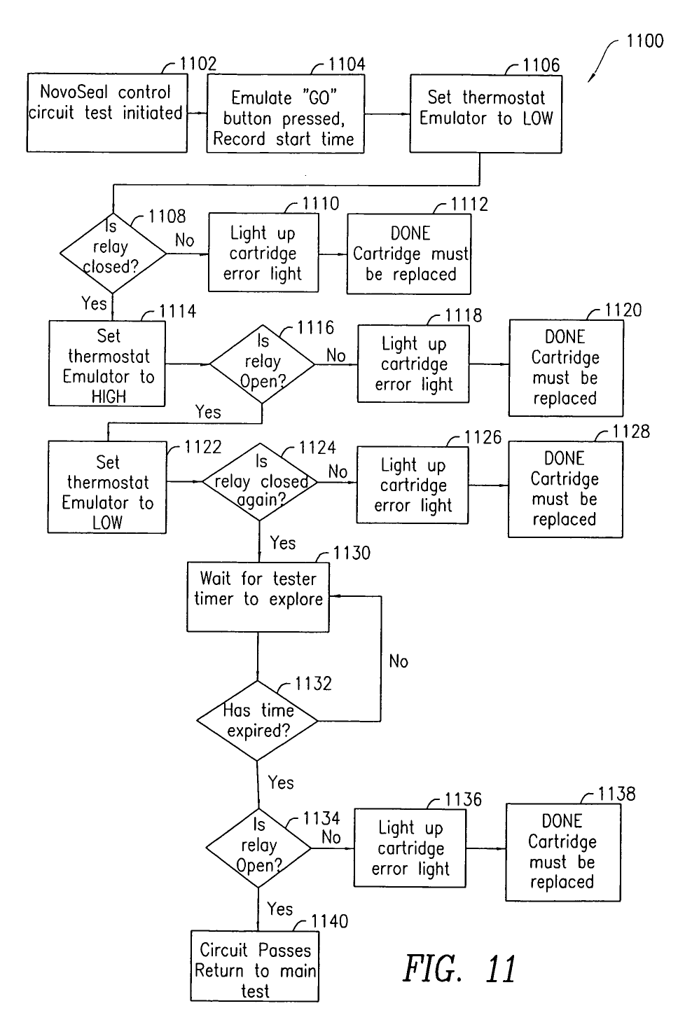

In some embodiments, the apparatus 100 can be used with a NovoSeal device, manufactured by Novomedics, LLC, Dickinson, Tex., where the NovoSeal device includes a catheter that can be inserted through the portal 118 on the apparatus 100. The catheter in the NovoSeal device can include a thermostat that measures temperature and generate a signal indicative of the measured temperature by adjusting its resistance to current flow. The same effect can be achieved by using the DigiPOT device 808, which is an electronically controllable resistance. The NovoSeal device includes a control circuit that can turn ON or OFF the heater disposed in the NovoSeal catheter. The control circuit does so by opening or closing a relay in the cartridge 210 that passes the current to the heater element in the catheter. Same effect can be achieved by determining whether or not the relay is open or closed by trying to signal through it and determining whether received sequence of signals matches the transmitted sequence of signals, which indicates that the relay is closed. If the sequences do not match, the relay is open. FIG. 11 shows a flow chart of an exemplary testing procedure for NovoSeal device's control circuit procedure.

For inter-integrated circuit (“12C”) serial bus commands, the charger base's microcontroller can be made into a slave when the cartridge microcontroller's signals to the image sensor are being tested. The base's microcontroller serves as a master when contents of the EEPROM memory device in the cartridge are read/written. As can be understood by one skilled in the art, the present invention's apparatus 100 can be used with the NovoSeal device or any other device that can be used during examination, visualization, sterilization, tissue-scarring, or any other procedure performed on the female genital tract. As such, the above reference to NovoSeal device is provided here for descriptive, exemplary, and non-limiting purposes only.

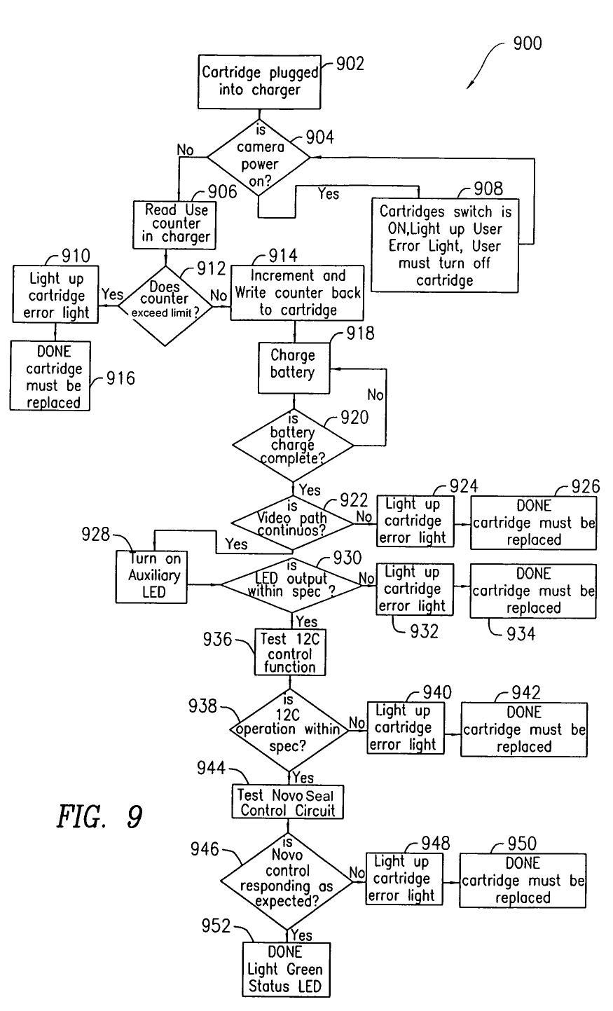

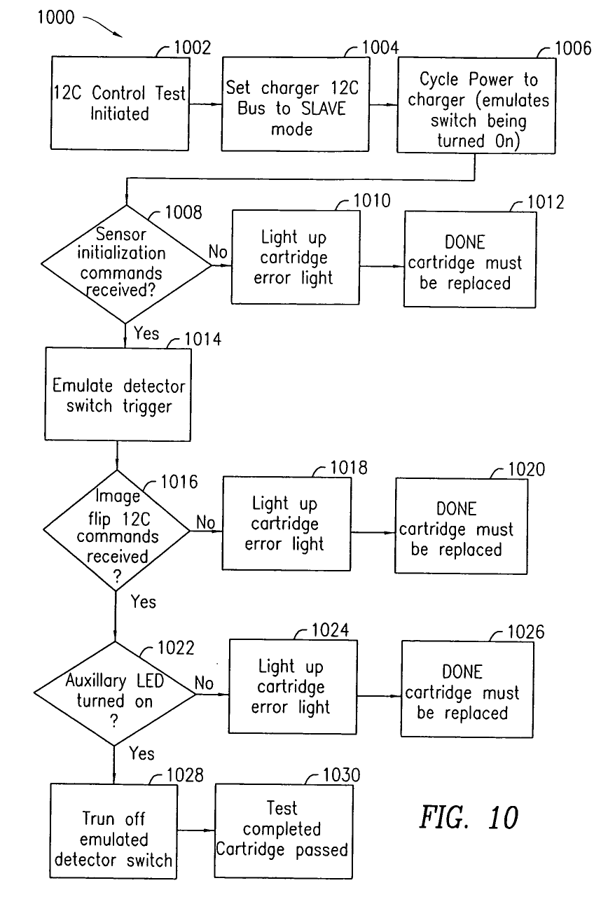

FIG. 9 is a flow chart illustrating an exemplary high level diagnostic procedure 900 for the cartridge 210 of the apparatus 100, according to some embodiments of the present invention. In step 902, the cartridge 210 is plugged into a charger. Then, in step 904, a determination is made whether the camera power is in the ON position. If it in the ON position, the cartridge switch is ON and the user error light is lit up indicating that the user should turn off the cartridge, as indicated in step 908. If the camera power is in the OFF position, then the process proceeds to step 906, wherein charger counter is read to determine how many times the battery in the cartridge has been recharged. In step 912, a determination is made as to whether the charger counter value has exceeded a predetermined value. If it has, then the process proceeds to step 910, where error light on the cartridge is lit up and the cartridge must be replaced, as indicated in step 916. If the counter has not exceeded the predetermined value, the counter value is incremented after the cartridge is plugged in, as indicated by step 914. Then, the battery in the cartridge is charged. (Step 918). In step 920, a determination is made whether the charging of the battery is complete. If not, the process returns to step 918 to continue charging the battery. If it is complete, then the process proceeds to step 922. In step 922, the video path is checked to determine if it is continuous. If not, the process proceeds to step 924 and 926, where error light is lit up (step 924) and cartridge is replaced (step 926). If it is continuous, the auxiliary LED is turned ON (Step 928) and its output is compared to specific predetermined values (e.g., as set by a manufacturer, a user, etc.) to determine whether such output equals to these values or is within a predefined range of values. (step 930). If it not, then the process proceeds to steps 932 and 934, where error light is lit up (step 934) and cartridge is replaced (step 936). If it is, then the process proceeds to step 938, where the 12C function is tested (see, FIG. 10) to determine whether it is operation within specification (e.g., is within a set of predetermined value as set by a manufacturer, a user, etc.). If not, steps 940 and 942 are performed, which are similar to steps 932 and 934, respectively. If it is, then control circuit of the device coupled to the apparatus 100 (e.g., NovoSeal device) is tested. (step 944). A determination is made whether the device's control circuit is operating as expected (e.g., its output is within a set of predetermined values as set by a manufacturer, a user, etc.). If not, a sequence of steps 948 and 950 is performed that is similar to steps 932 and 934, respectively. If it is, the testing procedure is completed and a green light is lit up on the cartridge 210. (Step 952).

FIG. 10 is a flowchart illustrating an I2C control testing procedure 1000, according to some embodiments of the present invention. In step 1002, the I2C control testing procedure is initiated. In step 1004, the charger's I2C bus is set to “SLAVE” mode. Then, the processing proceeds to step 1006, where power is cycled to the charger, which is equivalent to the detector switch being turned on. In step 1008, a determination is made whether the sensor commands are received. If not, the processing proceeds to steps 1010 and 1012, which are similar to steps 932 and 934 of FIG. 9, respectively. If the commands are received, then a procedure is performed (as described above with regard to FIG. 8) that emulates triggering of the detector switch. (Step 1014). The method then determines whether image flip commands have been received by the processor. If not, then steps 1018 and 1020 are performed, which are similar to steps 932 and 934 of FIG. 9, respectively. If the commands are received, then a determination of whether the auxiliary LED has been turned ON is performed. (Step 1022). If not, then steps 1024 and 1026 are performed indicating error and that the cartridge should be replaced. (similar to steps 932, 934, respectively). If the LED is ON, then switch is turned OFF and the procedure is completed. (Steps 1028 and 1030).

FIG. 11 is a flow chart of an exemplary NovoSeal control circuit testing procedure 1100, according to some embodiments of the present invention. As can be understood by one skilled in the art, the testing procedure 1100 can be performed for any device that is coupled to the apparatus 100 and is not limited to the NovoSeal device. In steps 1102-1106, the NovoSeal control circuit testing procedure is initiated (Step 1102), the actuation button 118 is pressed by the user, thereby initiating recording time by the camera (Step 1104), and thermostat in the control circuit is set to LOW (Step 1106). In step 1108, a determination is made whether control circuit's relay is closed. If it is not, then processing proceeds to steps 1110 and 1112, which are similar to steps 932 and 934, respectively. If it is closed, then thermostat is set to HIGH, as indicated by step 1114. The system then checks whether the relay is now open. (Step 1116). If it is closed, steps 1118 and 1120 are performed (similar to steps 932, 934, respectively). If it is open, then thermostat is set to LOW again. (Step 1122). The system then checks whether the relay has closed again in step 1124. If not, then processing proceeds to steps 1126 and 1128, which are similar to steps 932, 934, respectively. If it is closed, the system waits until the testing time expires. (Steps 1130-1132). Once the time has expired, the system checks whether the relay is open, as shown in step 1134. If not, steps 1136 and 1138 are performed indicating error and a need for cartridge replacement. If it is open, then an indication is provided affirming that the control circuit has passed the test. (Step 1140).

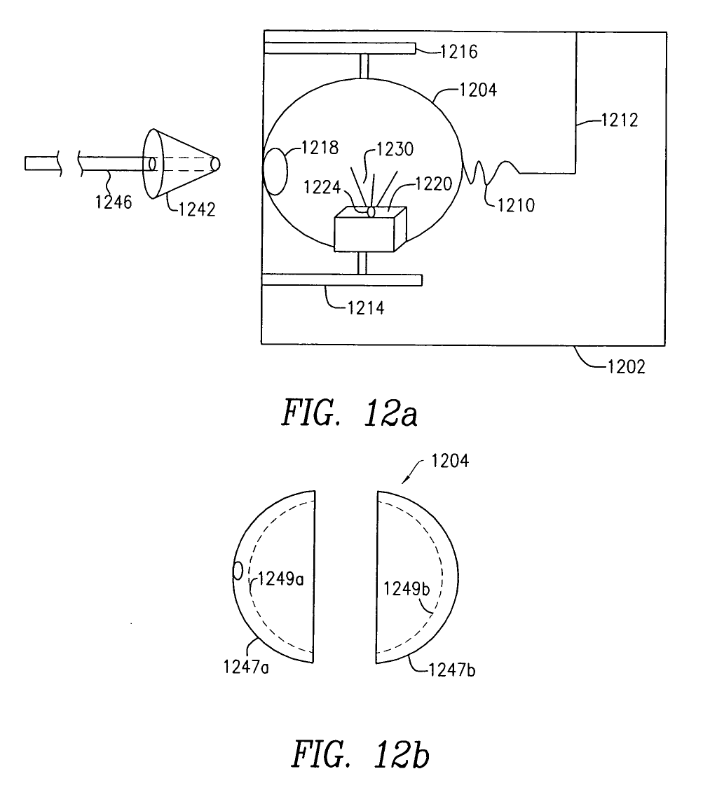

FIGS. 12a-b illustrate an alternative embodiment of a light source 1200 using an integrating sphere disposed within a cartridge, according to some embodiments of the present invention. The light source is disposed within a cartridge 1202 and includes an integrating sphere 1204 that is suspended on a spring 1210, coupled to the cartridge 1202 via a pin 1212, and rails 1214 and 1216, where the rails 1214 and 1216 are coupled to the cartridge 1202 as well. The cartridge 1202 is similar to the cartridge 210 discussed above. The spring 1210 allows the sphere to move back and forth on the rails 1214 and 1216 as desired. In some embodiments, such movement allows the sphere to maintain a specific distance from the fiber 1246 into which the sphere 1204 is configured to shine the light it generates. In some embodiments, the sphere 1204 can consist of a hollow interior coated with a reflective material (e.g., a plurality of mirrors) and composed of two halves 1247a and 1247b configured to be coupled together to form a uniform sphere 1204, as shown in FIG. 12b. The sphere 1204 can also include an entry for the wire connected to an LED 1220 disposed inside the sphere, where the LED 1220 is configured to emit light 1230 that is reflected inside the sphere 1204. The sphere 1204 also includes an opening 1218, which serves as an exit port for the light reflected by sphere's interior reflective surfaces. The LED 1220 can be configured to be heatsinked to the sphere 1204.

In some embodiments, the sphere 1204 can be configured to be inserted sideways into the cartridge 1202. This allows the sphere 1204 to slide back and forth inside the cartridge on the rails 1214 and 1216.

The fiber 1246 can be configured to include a cone shaped adaptor 1242 at its one end disposed near the light source that can be configured to be inserted into the opening 1218. The opening 1218 can include a rubber gasket to accommodate placement and proper securing of the cone-shaped adaptor 1242 to the opening 1218.

Some advantages of the apparatus 100 include providing visualization and sterilization using any known means. The apparatus 100 can use any heating devices for scarring the tissue and/or can share some components with such devices, which can include electronics (e.g., one device can supply power to the other device and vice versa; power provided by the devices can be constant (such as X volts and Y amps); and power can be modulated using some control information; power can be generally defined as energy transfer from one device to the other for the purpose of operating the second device). Additionally, the apparatus 100 and the heating device can provide control information to each other (e.g., signals that indicate start or end of a specific procedure (such as sterilization, visualization, etc.); signals that direct the other device to perform some action (such as heating, etc.)). The apparatus 100 and the other device can also share some mechanical components. For example, one device can depend on the other device for delivery or mechanical support. This can be applicable in case where the sterilization device does not have a handle and shaft of its own, but is rather built into the distal end of the visualization device. In some embodiments, the visualization device can have a means that emerge from the tip for the sterilization part of the procedure, and after completion of the sterilization procedure, retract back into the distal end of the visualization device. Other shared components can include the handle, the shaft or other mechanical features. Additionally, the devices can include mechanical features specifically designed to hold the other device in a specific location and/or to manipulate it. In commercial setting, the devices can be supplied in a single or multiple packages.

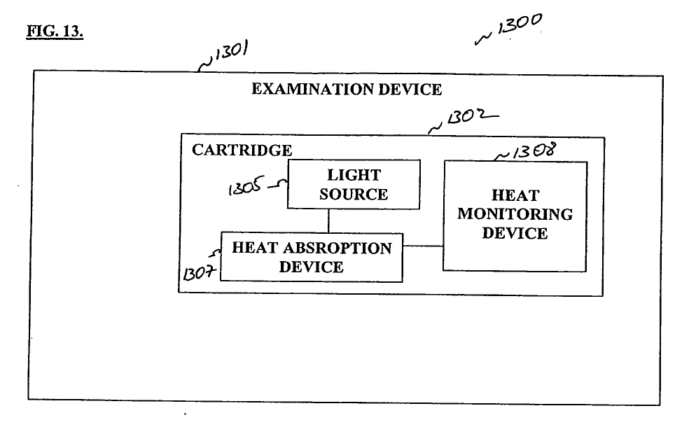

FIG. 13 is a block diagram of an exemplary heat monitoring system 1300, according to some embodiments of the present invention. As stated above, most endoscopic devices seeking to provide a built-in or external illumination source use a bright light source, such as an LED or Xenon bulb. Such bright light sources generate substantial amounts of heat during operation. The heat can cause damage to the user of the examination device (described above), the patient as well as external components of the device. In some devices, as discussed above, the heat can be expelled from the device using heat-sinks for heat absorption and/or fans to increase airflow, thereby cooling the device. In some conventional devices, however such heat-absorption or cooling devices are not feasible due to mechanical constraints.

As stated above, in some embodiments of the present invention an LED light source is disposed in the cartridge. Referring to FIG. 13, the system 1300 includes an examination device 1301 that includes, among other components (not shown in FIG. 13), a cartridge 1302. The cartridge 1302 includes an LED source 1305, a heat absorption device 1307, and a heat monitoring device 1308. These components can be powered by a power source (not shown in FIG. 13) and include any other electronics as well as any components shown and described with regard to FIGS. 1-12b above. As can be understood by one skilled in the art, the LED source 1305 can be substituted for any light generation device(s). As discussed above, the cartridge 1302 can be enclosed in a plastic chamber with no other means for heat escape but the heat absorption device 1307 that is coupled to the light source 1305. As can be understood by one skilled in the art, the heat absorption device can be any heat management device/heat sponge/heat sink that has a high thermal mass (for example, a big piece of copper) and is configured to absorb heat expelled by the light source 1305. As the heat absorption device 1307 absorbs the generated heat, it also gradually warms itself up.

In some embodiments, during operation of the device, the heat absorption device can warm up less than 10 degrees C. over 45 minutes of usage. However, in some embodiments, the heat absorption device 1307 can warm up more than and in some instances overheat. These situations include adjustment of the light source 1305 to be brighter. Such cases could potentially lead to dangerous situations where the heat absorption device is surrounded by plastic or other heat susceptible material that could melt and potentially injure the user and/or patient and damage/destroy components of the examination device 1301.

In some embodiments, in order to limit the risk of injury and/or such damage(s), a heat monitoring device 1308 is provided within the cartridge 1302. In some embodiments, the device 1308 can be disposed outside the cartridge 1302 and monitor the temperature, T_{A}, of the heat absorption device from the outside of cartridge. The device 1308 can also include an indicator that can be disposed on the housing of the examination device 1301. The device 1308 can be configured to warn the user that the temperature of the heat absorption device 1307 has reached a certain predetermined threshold temperature, T_{H}. Additionally, an audio and/or audio-visual alert can be used to alert the user. In some embodiments, the device 1308 can include circuitry that is configured to detect temperature T_{A} of the heat absorption device 1307 and using a microcontroller disposed within the circuitry of the device 1308 compare it to a preset threshold temperature T_{H}. If T_{A} is greater than T_{H}, then the device 1308 can be configured to shut down the light source 1305 to prevent further overheating of the device 1307 and a potential damage to the device 1301 and/or injury to user/patient. In some embodiments, the heat monitoring device 1308 can include a switching device that can cut off power to the light source 1305 in the event overheating is detected. In some embodiments, the heat monitoring device 1308 can periodically monitor the temperature T_{A} of the heat absorption device 1307. In some embodiments, the heat monitoring device 1308 can be configured to thermally shutdown in the cartridge 1302. If upon detection of the temperature T_{A} and comparison of this temperature to the threshold temperature T_{H}, T_{A} is less than T_{H}, the device 1301 can continue being used. In some embodiments, the heat monitoring device can be a thermistor or any other heat management device. The microcontroller can be configured to read the value of the thermistor and monitor the temperature of the heat absorption device 1305. The microcontroller can programmatically monitor the thermistor readout, and turn off the light source 1305 in the event the temperature exceeds a preset threshold level.

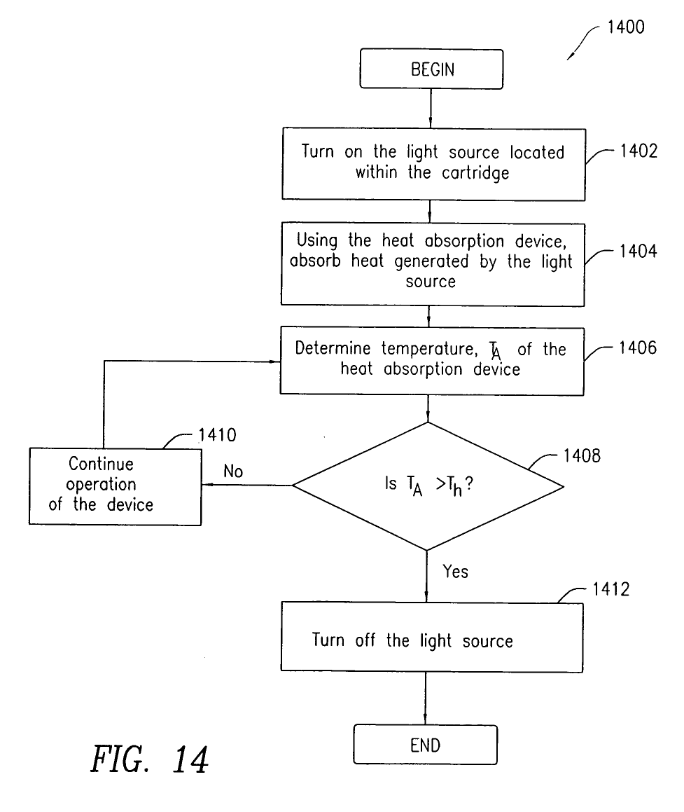

FIG. 14 illustrates an exemplary method 1400 for monitoring temperature of the heat absorption device within the examination device 1301, according to some embodiments of the present invention. In step 1402, the light source 1305 is turned on for the purposes of examination of a bodily cavity. At the same time, the heat monitoring device 1308 can be also switched on to begin monitoring the temperature of the heat absorption device 1307. As indicated in step 1404, the heat generated by the light source 1305 is being absorbed by the heat absorption device 1307. At the same time, the heat monitoring device can continue monitoring the temperature of the heat absorption device.

In step 1406, temperature T_{A} of the heat absorption device 1307 is determined. The heat monitoring device 1308 compares T_{A} to a preset threshold temperature T_{H}. (Step 1408). If the T_{A} is greater than T_{H}, then the light source 1305 is shut down to prevent further generation of heat. (Step 1412). If not, then operation of the device is continued. (Step 1410).

As can be understood by one skilled in the art, the threshold temperature T_{H} can be preset by the manufacturer or can be predetermined by the user according to various operating parameters, e.g., length of time during which the device will be used, power output of the light source, voltage level, operational power of other components, and/or physiological parameters of the patient being examined. As can be further understood by one skilled in the art, the light source can be any energy source.

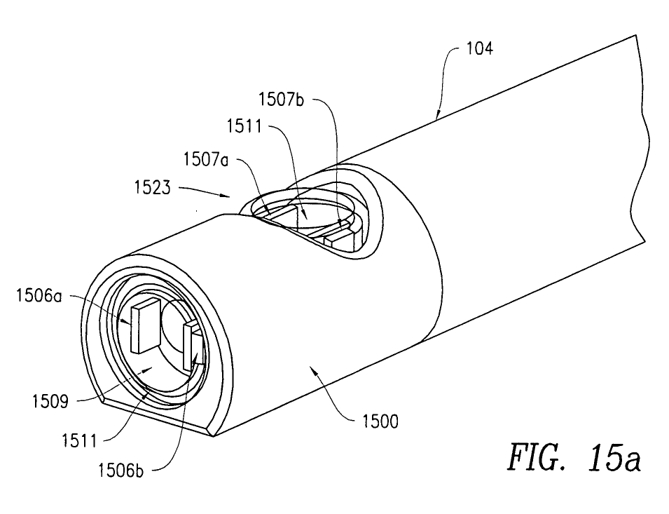

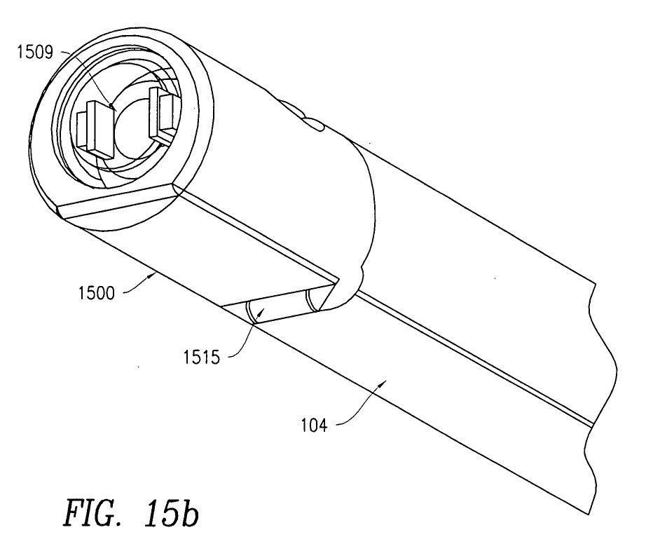

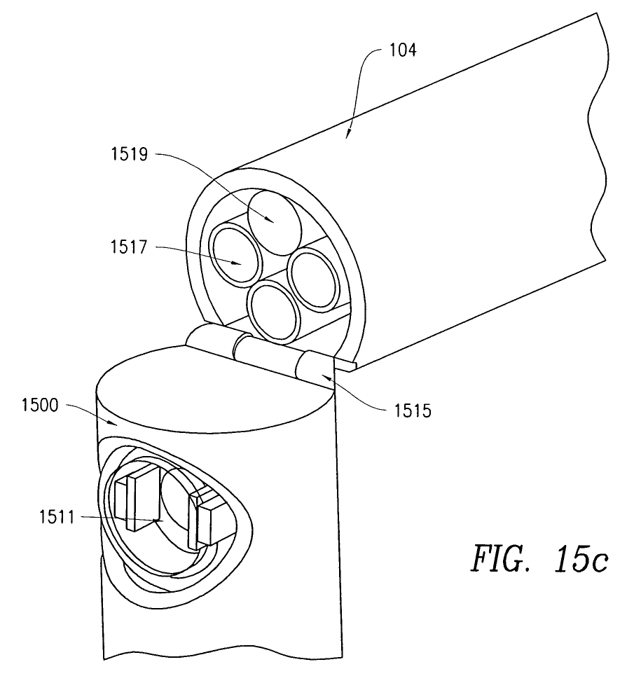

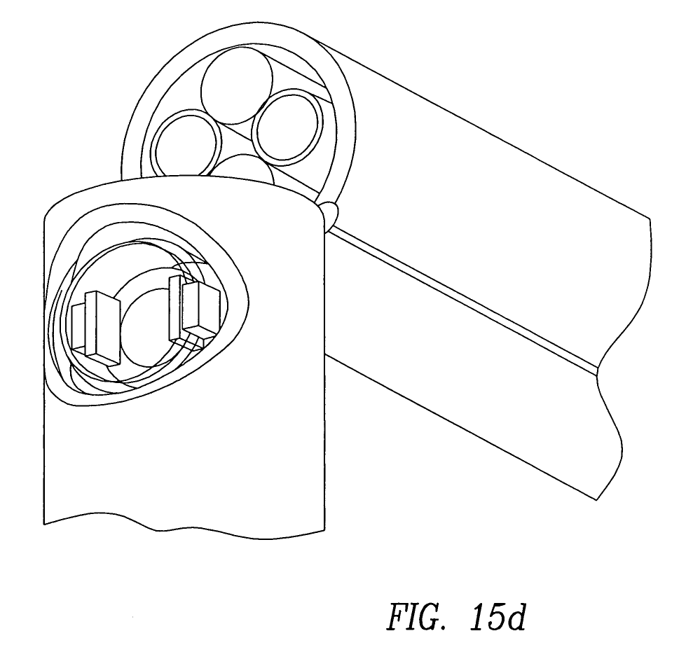

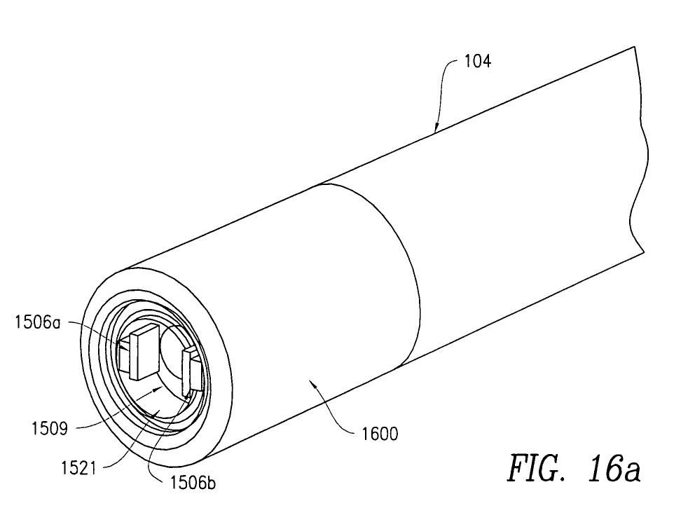

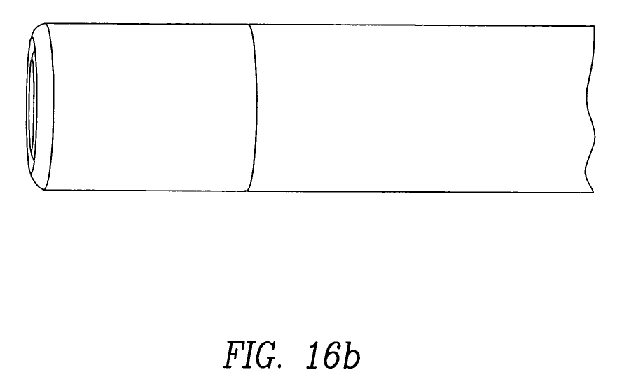

FIGS. 15a-d illustrate another exemplary configuration of a tip for the device 100, according to some embodiments of the present invention. Referring to FIGS. 15b-2d, the tip 1500 is coupled to the shaft 104 and is configured to pivot about a hinged portion 1515 to open interior components of the shaft 104 and during examination of internal bodily cavities. The tip 1500 further includes a first camera portion 1509, first LED sources 1506a and 1506b, and a second camera portion 1511, second LED sources 1507a and 1507b. The first camera portion 1509 and LED sources 1506 are configured to be disposed substantially adjacent the distal end of the tip 1500. The second camera portion 1511 and second LED sources 1507 are configured to be disposed in a sidewall of the tip 1500, as shown in FIG. 15a. The shaft 104 can also be provided with saline channels 1517 for inflow and outflow of a saline solution as well as a power/video/image cable 1519. The first LED sources 1506 are configured to provide light to the first camera 1509 when the tip 1500 is inserted into a bodily cavity of the patient. Similarly, the second LED sources 1507 are configured to provide light to the second camera 1511. The first and second cameras 1509, 1511 are configured to provide a visual of the interior of the bodily cavity. Having two camera devices, a user of the device is able to have a stereoscopic view of the bodily cavity. Further, the camera devices can be configured to assist the user during advancement of the device into a bodily cavity. In some embodiments, both camera devices can be protected by respective protector lenses 1521 and 1523. In some embodiments, the tip 1500's camera device are connected to the housing of the device using video/power/image cable(s) 1519 (not shown in FIGS. 15a-d). Each camera may have its own processing circuit and/or printed circuit board (“PCB”) and be connected to a separate processor in the housing 102. In some embodiments, a single processor is configured to process information from both cameras. The cameras can share a single power source or have separate power sources.

As shown in FIGS. 15c and 15d, the tip 1500 is rotated about its hinged portion 1515 to expose interior of the shaft 104. In some embodiments, the tip 1500 can be rotated 180 degrees and configured to allow cameras 1509 and 1511 to face in opposite directions as compared to their original positions. For examiner, camera 1509 initial position is facing forward away from the handle portion, while after rotation it faces backward toward the handle portion. The initial position of the camera 1511 is facing upward, while after 180-degree rotation it can be configured to face downward. The shaft portion 104 and the distal tip 1500 can be configured to have coinciding flattened portions, as shown in FIGS. 15a-2d, that allow for a full 180-degree rotation. The shaft may contain saline channels, channels that are configured for insertion of tools (e.g., surgical tools) or any other channels. While the interior of the shaft is open, the second camera device 1511 is configured to provide the user with a visual information in front of the opened shaft 106. Such information can assist the user (a doctor or any other medical professional) during medical procedures (whether during medical examinations, surgical procedures or otherwise). Once the procedure is completed, the tip can be closed and the device removed from the bodily cavity. Opening and closing of the tip 1500 can be controlled via push and/or pull wires coupled inside the housing 102. In some embodiments, upon pulling of the wire, the tip can be closed and upon pushing of the wire, the tip can be opened. In some embodiments, while the tip is opened, the first camera along with the second camera can continue to provide the user with a stereoscopic view of the cavity being examined. In some embodiments, a device 100 can be so programmed as to process images from both cameras and provide a combined imagery to the user.

In some embodiments, the device 100 can be configured to be used for examination of female genital tract. As such, while inserting the tip 1500, the wall of the cervical canal can be examined by the user while passing though it.

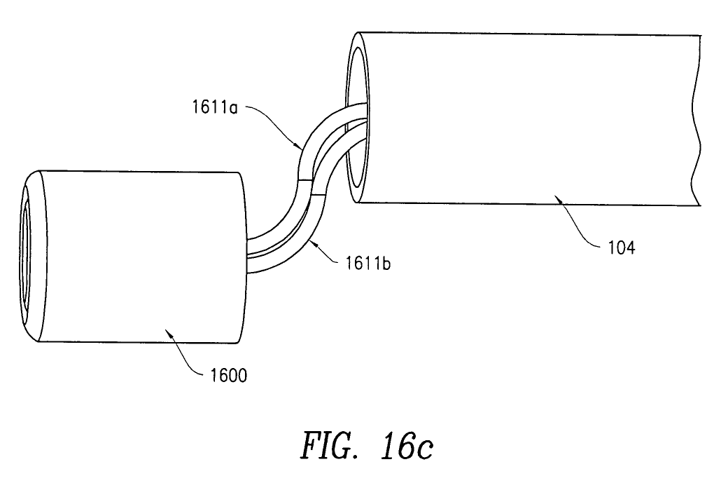

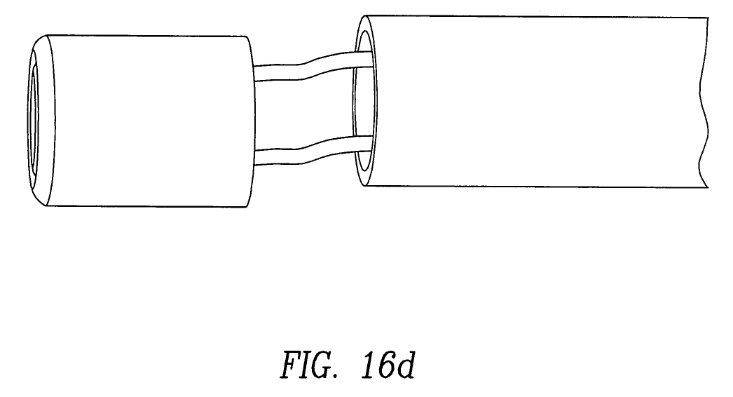

FIGS. 16a-d illustrate another configuration of the tip 1600, according to some embodiments of the present invention. The tip 1600 can be configured to be similar to the tip 106 or the tip 1500 discussed above (i.e., it may have one camera disposed at its distal end or have more than one camera disposed at its distal end and one disposed in a sidewall of the tip). As shown in FIGS. 16c and 16d, the tip 1600 can be separated from the shaft 106 using two wires 1611a and 1611b that are configured to protrude from the interior of the shaft 106 and connected to the tip 1600. In some embodiments, the tip 1600 can be hingedly secured to the two wires 1611 so that the tip 1600 can be rotated and translated once it is separated from the shaft 106, thus, giving the user a substantially 360-degree view of the cavity being examined. In some embodiments, the wires 1611 can be metallic, plastic or any other material having the following characteristics: formable to a certain shape; flexible all the way back to a straight line; capable of retaining its formed shape after being held straight for a long time. In some embodiments, the wires 1611 can be configured to provide the user with an ability to turn the camera from a forward view to almost all the way back and then all the way back to forward again. In some embodiments, this motion can be combined with a longitudinal rotation of the shaft to provide the user with a complete picture of the interior of the bodily cavity being examined (e.g., uterus), thereby allowing the user to visualize the entire interior of a much smaller space.

Other exemplary configurations of the tip are disclosed in the co-owned, co-pending U.S. patent application Ser. No. 11/975,409 to Sanders et al., filed Oct. 19, 2007, and entitled “Optical Surgical Device and Methods of Use”, the disclosure of which is incorporated by reference in its entirety. In view of the different features of the embodiments of the apparatus disclosed herein, a person skilled in the art will readily appreciate that different embodiments of the method of use of the optical apparatus of the present invention are also possible, in accordance with the specific features of the optical device in the related embodiments. Such alternative embodiments of the method of use are all within the scope and spirit of the present invention.

In view of the different features of the embodiments of the apparatus disclosed herein, a person skilled in the art will readily appreciate that different embodiments of the method of use of the optical apparatus of the present invention are also possible, in accordance with the specific features of the optical device in the related embodiments. Such alternative embodiments of the method of use are all within the scope and spirit of the present invention.

Thus it is seen that apparatus for examining a body cavity and methods of use are provided. Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

What is claimed:

1. An optical apparatus comprising:
a housing including a shaft portion, and a handle portion extending from a proximal end of the shaft portion and defining a cavity that contains an output connector;
a camera assembly coupled to a distal end of the shaft, the camera assembly comprising a camera; and
a removable cartridge having a limited number of uses and is receivable within the cavity and including an input connector matable with the output connector, an image processing engine, a power source, a light source, and a heat-absorption device for absorbing heat generated by the light source, wherein the light source is configured to direct light toward the input connector and illuminate a field of view for the camera.

2. The apparatus according to claim 1, wherein the light source is a light emitting diode (“LED”).

3. The apparatus according to claim 1, wherein the shaft further comprises a rotatable tip coupled to a distal end of the shaft portion, wherein the rotatable tip further accommodates placement of the camera.

4. The apparatus according to claim 1, wherein the light source is an integrating sphere having an LED disposed on an interior portion of the integrating sphere and configured to generate light;
the sphere having
an interior coated with a reflective material configured to internally reflect light generated by the LED;
an opening through which light internally reflected by the integrating sphere is configured to exit toward the input connector.

5. The apparatus according to claim 4, wherein the integrating sphere is configured to be slidably coupled to the removable cartridge.

6. The apparatus according to claim 3, further comprising a saline channel connector for delivery of saline fluid during a visualization/sterilization procedure of a female genital tract of a patient, during which
the saline channel connector is configured to be connected to an external saline delivery device;
the shaft portion is inserted into the uterus of the patient, wherein the tip is first inserted into the uterus;
the tip is opened;
saline fluid is delivered via the saline channel connector to clear a field of view for the camera;
fallopian tubes of the patient are located;
an external sterilization catheter is configured to be inserted and activated, wherein after completion of a sterilization procedure using the external sterilization catheter, the external sterilization catheter is configured to be removed;
when the external sterilization catheter is removed, the tip and the shaft portion are removed from the female genital tract.

7. The apparatus according to claim 3, further comprising a treatment device for treating tissue in the female genital tract of a patient.

8. The apparatus according to claim 7, wherein the treatment device is a heating device configured to scar the tissue, wherein the heating device is configured to operate at a predetermined temperature and during a predetermined period of time.

9. The apparatus according to claim 1, wherein the removable cartridge is configured to be rechargeable using a charging device.

10. The apparatus according to claim 9, wherein the cartridge is configured to be recharged a limited number of times.

11. The apparatus according to claim 10, wherein the charging device includes a counter configured to be adjusted every time the cartridge is charged.

12. The apparatus according to claim 11, wherein the charging device is configured to recharge the cartridge when the camera is not operating.

13. The apparatus according to claim 12, wherein the charging device is configured to recharge the cartridge when the light source is not operating.

14. An optical apparatus comprising:
a housing including a shaft portion, and a handle portion extending from a proximal end of the shaft portion and defining a cavity that contains an output connector;
a camera assembly coupled to a distal end of the shaft, the camera assembly comprising a camera; and
a removable cartridge having a limited number of uses and is receivable within the cavity and including
an input connector matable with the output connector;
an image processing engine;
a power source;
a light source;
a heat-absorption device for absorbing heat generated by the light source;
a heat-monitoring device for monitoring temperature of the heat-absorption device during operation of the removable cartridge;
wherein the light source is configured to direct light toward the input connector and illuminate a field of view for the camera.

15. The apparatus according to claim 14, wherein the light source is a light emitting diode (“LED”).

16. The apparatus according to claim 15, wherein the heat monitoring device is configured to turn off the light source when a temperature of the heat absorption device exceeds a predetermined threshold temperature.

17. The apparatus according to claim 16, wherein the heat monitoring device is configured to periodically determine temperature of the heat absorption device and compare the determined temperature to the predetermined threshold temperature.

18. The apparatus according to claim 17, wherein the predetermined threshold temperature is adjustable.

19. The apparatus according to claim 15, wherein the shaft further comprises a rotatable tip coupled to a distal end of the shaft portion, wherein the rotatable tip further accommodates placement of the camera.

20. The apparatus according to claim 19, further comprising a saline channel connector for delivery of saline fluid during a visualization/sterilization procedure of a female genital tract of a patient, during which the saline channel connector is configured to be connected to an external saline delivery device;
the shaft portion is inserted into the uterus of the patient, wherein the tip is first inserted into the uterus;
the tip is opened;
saline fluid is delivered via the saline channel connector to clear a field of view for the camera;
fallopian tubes of the patient are located;
an external sterilization catheter is configured to be inserted and activated, wherein after completion of a sterilization procedure using the external sterilization catheter, the external sterilization catheter is configured to be removed;
when the external sterilization catheter is removed, the tip and the shaft portion are removed from the female genital tract.

21. The apparatus according to claim 19, further comprising a treatment device for treating tissue in the female genital tract of a patient.

22. The apparatus according to claim 21, wherein the treatment device is a heating device configured to scar the tissue, wherein the heating device is configured to operate at a predetermined temperature and during a predetermined period of time.

23. The apparatus according to claim 22, wherein the removable cartridge is configured to be rechargeable using a charging device.

24. The apparatus according to claim 23, wherein the cartridge is configured to be recharged a limited number of times.

25. The apparatus according to claim 24, wherein the charging device includes a counter configured to be adjusted every time the cartridge is charged.

26. The apparatus according to claim 25, wherein the charging device is configured to recharge the cartridge when the camera is not operating.

27. The apparatus according to claim 26, wherein the charging device is configured to recharge the cartridge when the light source is not operating.

28. A method for examining a bodily cavity using an optical apparatus having
a housing including a shaft portion, and a handle portion extending from a proximal end of the shaft portion and defining a cavity that contains an output connector;
a camera assembly coupled to a distal end of the shaft, the camera assembly comprising a camera; and
a removable cartridge having a limited number of uses and is receivable within the cavity and including
an input connector matable with the output connector;
an image processing engine;
a power source;
a light source;
a heat-absorption device for absorbing heat generated by the light source;
a heat-monitoring device for monitoring temperature of the heat-absorption device during operation of the removable cartridge;
wherein the light source is configured to direct light toward the input connector and illuminate a field of view for the camera;
the method comprising the steps of:
using the light source, examining the bodily cavity;
using the heat absorption device, absorbing heat generated by the light source during examination of the bodily cavity;
determining a temperature of the heat absorption device;
comparing the determined temperature to a predetermined threshold temperature; and,
turning off the light source when the determined temperature exceeds the predetermined threshold temperature.

29. The method according to claim 28, wherein the light source is a light emitting diode (“LED”).

30. The method according to claim 29, further comprising the step of:
periodically determining the temperature of the heat absorption device and comparing the determined temperature to the predetermined threshold temperature.

31. The method according to claim 30, wherein the predetermined threshold temperature is adjustable.

32. An optical apparatus comprising:
a housing including a shaft portion, and a handle portion extending from a proximal end of the shaft portion and defining a cavity that contains an output connector;
a distal tip portion configured to be pivotally coupled to a distal end of the shaft portion;
a first camera assembly coupled to a distal tip of the shaft, the first camera assembly comprising a first camera and a first light source, wherein the first camera assembly is disposed at a distal end of the distal tip;
a second camera assembly coupled to a distal tip of the shaft, the second camera assembly comprising a second camera and a second light source, wherein the second camera assembly is disposed in a sidewall of the distal tip;
wherein the first and second light source are configured to illuminate respective fields of view for the first and second camera.

33. The apparatus according to claim 32, wherein the light sources are light emitting diodes (“LEDs”).

34. The apparatus according to claim 33, wherein the field of view of the first camera is configured to be disposed at an angle to the field of view of the second camera.

35. The apparatus according to claim 34, wherein upon pivoting of the distal tip, an interior portion of the shaft portion is configured to be exposed.

36. The apparatus according to claim 35, wherein the shaft portion is configured to include a channel configured to allow insertion of surgical tools.

37. The apparatus according to claim 36, wherein the shaft portion further comprising a saline channel for delivery of saline fluid during a visualization/sterilization procedure.

38. A method for examining a bodily cavity using an optical apparatus having
a housing including a shaft portion, and a handle portion extending from a proximal end of the shaft portion and defining a cavity that contains an output connector;
a distal tip portion configured to be pivotally coupled to a distal end of the shaft portion;
a first camera assembly coupled to a distal tip of the shaft, the first camera assembly comprising a first camera and a first light source, wherein the first camera assembly is disposed at a distal end of the distal tip;
a second camera assembly coupled to a distal tip of the shaft, the second camera assembly comprising a second camera and a second light source, wherein the second camera assembly is disposed in a sidewall of the distal tip;
wherein the first and second light source are configured to illuminate respective fields of view for the first and second camera.
the method comprising the steps of:
inserting the device into a bodily cavity;
pivoting the distal tip away from the shaft portion; and,
using at least one or both of the first and second light sources and at least one or both of first and second cameras, examining the bodily cavity.

39. The method according to claim 38, wherein the light source is a light emitting diode (“LED”).

40. An optical apparatus comprising:
a housing including a shaft portion, and a handle portion extending from a proximal end of the shaft portion and defining a cavity that contains an output connector;
a distal tip portion configured to be removably coupled to a distal end of the shaft portion;
a camera assembly coupled to the distal tip of the shaft, the camera assembly comprising a camera and a light source, wherein the camera assembly is disposed at a distal end of the distal tip;
wherein the light source is configured to illuminate a field of view for the camera.

41. The apparatus according to claim 40, wherein the distal tip is configured to be extended away from the shaft portion using a flexible wiring mechanism.

42. The apparatus according to claim 41, wherein upon being extended away from shaft portion, the distal tip is configured to be rotatable and/or translatable about at least one location where the distal tip connects to the flexible wiring mechanism and/or location where the flexible wiring mechanism connects to the distal end of the shaft portion.

43. The apparatus according to claim 42, wherein upon retraction of the distal tip toward the shaft portion, the flexible wiring mechanism is configured to retract into an interior of a distal end of the shaft portion.

44. A method for examining a bodily cavity using an optical apparatus having
a housing including a shaft portion, and a handle portion extending from a proximal end of the shaft portion and defining a cavity that contains an output connector;
a distal tip portion configured to be removably coupled to a distal end of the shaft portion;
a camera assembly coupled to the distal tip of the shaft, the camera assembly comprising a camera and a light source, wherein the camera assembly is disposed at a distal end of the distal tip;
wherein the light source is configured to illuminate a field of view for the camera;
the method comprising the steps of:
inserting the device into a bodily cavity;
extending the distal tip away from the shaft portion; and,
using the light source and the camera, examining the bodily cavity.
